(19) United States
(12) Patent Application Publication
(10) Pub. No.: US 2006/0141490 A1
(43) Pub. Date: Jun. 29, 2006

(54) MyD88 as a therapeutic target for cancer

(76) Inventors: Isabelle Coste-Invernizzi, Chazay d'Azergues (FR); Toufic Renno, Civrieux d'Azergues (FR)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/182,962

(22) Filed: Jul. 15, 2005

Related U.S. Application Data
(60) Provisional application No. 60/589,122, filed on Jul. 19, 2004.

(51) Int. Cl. C12Q 1/68; G01N 33/574

(52) U.S. Cl. 435/6; 435/7.23

(57) ABSTRACT

The invention relates to methods for identifying anti-cancer agents using MyD88 as a therapeutic target.

### CROSS-REFERENCE TO RELATED APPLICATIONS

This application claims priority under 35 U.S.C. § 119(e) to U.S. application Ser. No. 60/589,122.

### BACKGROUND OF THE INVENTION

1. Field of the Invention

The invention relates to methods for identifying anti-cancer agents using MyD88 as a therapeutic target.

### 2. BACKGROUND

Cancer is one of the leading causes of death in the world. Therefore, it is essential that we develop new methods to treat this deadly disease. Many current cancer therapies affect rapidly dividing cells. These therapies have devastating side effects because they affect all rapidly dividing cells, such as cells of the gastrointestinal tract and hair follicles, and not just cancer cells. Therefore, new methods of treatment are needed that do not have such devastating side effects. The present application identifies MyD88 as a therapeutic target in the treatment of cancer.

Mammalian MyD88 is an adaptor protein in the signal transduction pathway mediated by interleukin-1 (IL-1) receptors and Toll-like receptors (TLRs). TLRs and receptors of the IL-1 family interact with the protein MyD88. Specifically, the carboxy-terminal Toll/IL-1 receptor (TIR) domain of MyD88 interacts with the cognate domains in the intracytoplasmic tails of the TLRs. Then, MyD88 interacts with the Pelle-related kinases of the IRAK (IL-1 receptor associated kinase) family. Specifically, the amino-terminal death domain of MyD88 mediates interaction with the corresponding domain of IRAK. A result of these interactions is the activation of the Rel transcription factor NF-κB, which results in the activation of inflammatory response genes.

### SUMMARY OF THE INVENTION

An embodiment of the invention provides a method for identifying an anti-cancer agent comprising: a) contacting a test sample with a cell that expresses MyD88 or a biologically active fragment thereof; and b) measuring MyD88 expression level in said cell, whereby the test sample is identified to contain an anti-cancer agent if the test sample causes decreased MyD88 expression when compared to a control cell. The contacting step may comprise introducing the sample into the cell.

An alternative embodiment of the invention provides a method for identifying an anti-cancer agent comprising: a) contacting a test sample with a cell having a vector comprising a nucleic acid encoding MyD88 or a biologically active fragment thereof; and b) measuring MyD88 expression level in said cell, whereby the test sample is identified to contain an anti-cancer agent if the test sample causes decreased MyD88 expression when compared to a control cell. The contacting step may comprise introducing the sample into the cell.

Another embodiment of the invention provides a method for identifying an anti-cancer agent comprising: a) contacting a test sample with a cell having a vector comprising a nucleic acid encoding MyD88 or a biologically active fragment thereof that is operably linked to a reporter gene; and measuring MyD88 expression level in said cell, whereby the test sample is identified to contain an anti-cancer agent if the test sample causes decreased MyD88 expression when compared to a control cell. The contacting step may comprise introducing the sample into the cell.

### DETAILED DESCRIPTION OF THE INVENTION

All publications cited herein are incorporated by reference in their entirety.

### DEFINITIONS

The term “agent” means any molecule or compound, such as a small molecule (an organic molecule), an antibody or antibody fragment, siRNA, an antisense nucleic acid, a ribozyme, a polypeptide, DNA and RNA.

The term “antibody” means an immunoglobulin, i.e., containing two F_{ab} fragments connected to a F_{c}fragment. The term “antibody” includes polyclonal, monoclonal, chimeric, primatized, humanized and human antibodies. The term “antibody” includes any one of the five major classes of immunoglobulins: IgA, IgD, IgE, IgG and IgM, and also subclasses (isotypes) of immunoglobulins, i.e., IgG1, IgG2, IgG3, IgG4, IgA and IgA2.

The term “antibody fragment” means any fragment or combination of fragments of an immunoglobulin, such as F_{ab}, F_{c}, F_{(ab)2} and Fᵥ fragments.

The term “biologically active fragment” means a fragment of a protein that has full or partial biological activity of the entire protein.

The term “cancer” describes the physiological condition that is typically characterized by unregulated cell growth. Examples of cancer include, but are not limited to, carcinoma, lymphoma, blastoma, sarcoma and leukemia. More specific examples include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, prostate cancer, vulval cancer, thyroid cancer, hepatic carcinoma and various types of head and neck cancers.

The term “chemotherapeutic compound” means a chemical compound useful in the treatment of cancer.

The term “control cell” means a cell that expresses MyD88, but has not been contacted with a test sample.

The term “treatment” means therapeutic, prophylactic or suppressive measures for a disease or disorder leading to any clinically desirable or beneficial effect, including, but not limited to, alleviation of one or more symptoms, regression, and slowing or cessation of progression of the disease or disorder.

The term “siRNA” means short interfering RNA.

The terms “express”, “expresses”, “expression” and “expressing” all mean the transcription and translation of a nucleic acid to produce a polypeptide. In a cell, this means that the polypeptide will either be secreted, remain in the cytoplasm, or reside at least partially in the cell membrane.

The term “ligand” means any molecule that is capable of specifically binding to another molecule. A “ligand” can be, for example, a small molecule (an organic molecule), an antibody or antibody fragment, siRNA, an antisense nucleic acid, a polypeptide, DNA and RNA.

The term “patient” means both human and non-human animals.

The term “test sample” means a sample that includes an agent to be tested for its ability to decrease expression levels of MyD88.

The term “therapeutically effective amount” means an amount of an agent that will ameliorate one or more of the parameters that characterize medical conditions, diseases or disorders.

The terms “effective amount” and “amount effective” mean an amount of a pharmaceutical composition, such as an agent, that will cause a certain effect, such as an anti-cancer effect.

MyD88 Characterization

The nucleotide sequence of the complete open reading frame of the human MyD88 gene can be found in the GenBank® database accession no. BC013589. In addition, the amino acid sequence of human MyD88 can be found in the GenBank® database as accession no. AAH13589.

A person having skill in the art will, given the nucleic acid and amino acid sequence of MyD88, be able to produce any MyD88 protein or fragment thereof, antibody to the protein or fragment, nucleic acid or fragment thereof, nucleic acid probe, antisense, siRNA, etc. using standard molecular biology techniques. These molecules can then be used to provide a cell having a vector comprising a nucleic acid encoding MyD88 or a fragment thereof.

The present invention is based, in part, on the discovery that almost no MyD88−/−mice developed tumors when subjected to a DMBA/TPA two-stage, ras-dependent skin carcinogenesis protocol.

Contacting a Test Sample with a Cell that Expresses MyD88

A step of the method of the invention involves contacting a test sample with a cell that expresses MyD88 or a biologically active fragment thereof. The contacting step involves bringing the test sample and the cell into close physical proximity such that the sample and cell may contact each other. The contacting step will allow the sample to enter into the cell so that the sample's effect on the expression level of MyD88 can be determined. Alternatively, the sample may be introduced directly into the cell. For example, the sample may be introduced into the cell by electroporation, injection, transduction or transfection.

A Cell that Expresses MyD88 or a Biologically Active Fragment Thereof

A step of the method of the invention involves a cell that expresses MyD88 or a biologically active fragment thereof. The cell may be any kind of cell that expresses MyD88, but is preferably a mammalian cell. The cell may be a naturally occurring cell or may be a cell from a cell line. In addition, the cell may contain and express a vector comprising a nucleic acid encoding MyD88 or a biologically active fragment thereof. Alternatively, the cell may contain and express a vector comprising a nucleic acid encoding MyD88 or a biologically active fragment thereof that is operably linked to a reporter gene.

Polypeptides

As used herein, the term “polypeptide” or “peptide” means a fragment or segment, e.g., of MyD88, containing at least 8, preferably at least 12, more preferably at least 20, and most preferably at least 30 or more contiguous amino acid residues, up to and including the total number of residues in a complete protein. Polypeptides can comprise any part of the complete sequence of a protein. Thus, polypeptides could be produced by proteolytic cleavage of an intact protein, by chemical synthesis or by the application of recombinant DNA technology. In addition, the term “polypeptide” also includes additions, substitutions, deletions, and variants of a protein. The polypeptides, either alone or cross-linked or conjugated to a carrier molecule to render them more immunogenic, are useful as antigens to elicit the production of antibodies. The antibodies can be used, e.g., in immunoassays of the intact protein, for immunoaffinity purification, etc.

Nucleic Acids and Expression Vectors

As used herein, the term “isolated nucleic acid” means a nucleic acid, such as a RNA or DNA molecule, or a mixed polymer, which is substantially separated from other components that are normally found in cells or in recombinant DNA expression systems. These components include, but are not limited to, ribosomes, polymerases, serum components, and flanking genomic sequences. The term thus embraces a nucleic acid that has been removed from its naturally occurring environment, and includes recombinant or cloned DNA isolates and chemically synthesized analogs or analogs biologically synthesized by heterologous systems. A substantially pure molecule includes isolated forms of the molecule.

An isolated nucleic acid will generally be a homogeneous composition of molecules but may, in some embodiments, contain minor heterogeneity. Such heterogeneity is typically found at the ends of nucleic acid coding sequences or in regions not critical to a desired biological function or activity.

A “recombinant nucleic acid” is defined either by its method of production or structure. Some recombinant nucleic acids are made by the use of recombinant DNA techniques, which involve human intervention, either in manipulation or selection. Others are made by fusing two fragments that are not naturally contiguous to each other. Engineered vectors are encompassed, as well as nucleic acids comprising sequences derived using any synthetic oligonucleotide process.

For example, a wild-type codon may be replaced with a redundant codon encoding the same amino acid residue or a conservative substitution, while at the same time introducing or removing a nucleic acid sequence recognition site. Similarly, nucleic acid segments encoding desired functions may be fused to generate a single genetic entity encoding a desired combination of functions not found together in nature. Although restriction enzyme recognition sites are often the targets of such artificial manipulations, other site-specific targets, e.g., promoters, DNA replication sites, regulation sequences, control sequences, or other useful features may be incorporated by design. Sequences encoding epitope tags for detection or purification as described above may also be incorporated.

A nucleic acid “fragment” is defined herein as a nucleotide sequence comprising at least 17, generally at least 25, preferably at least 35, more preferably at least 45, and most preferably at least 55 or more contiguous nucleotides.

This invention further encompasses recombinant DNA molecules and fragments having sequences that are identical or highly homologous to those described herein. The nucleic acids of the invention may be operably linked to DNA segments that control transcription, translation, and DNA replication.

Nucleic acids encoding a protein, such as MyD88, or fragments thereof can be prepared by standard methods. For example, DNA can be chemically synthesized using, e.g., the phosphoramidite solid support method of Mafteucci et al. [J. Am. Chem. Soc. 103:3185 (1981)], the method of Yoo et al. [J. Biol. Chem. 764:17078 (1989)], or other well known methods. This can be done by sequentially linking a series of oligonucleotide cassettes comprising pairs of synthetic oligonucleotides.

Of course, due to the degeneracy of the genetic code, many different nucleotide sequences can encode MyD88. The codons can be selected for optimal expression in prokaryotic or eukaryotic systems. Such degenerate variants are of course also encompassed for use in this invention.

Moreover, nucleic acids encoding MyD88 can readily be modified by nucleotide substitutions, nucleotide deletions, nucleotide insertions, and inversions of nucleotide stretches. Such modifications result in novel DNA sequences that encode antigens having immunogenic or antigenic activity in common with the wild-type protein. These modified sequences can be used to produce wild type or mutant proteins, or to enhance expression in a recombinant DNA system.

Insertion of the DNAs encoding MyD88 into a vector is easily accomplished when the termini of both the DNA and the vector comprise compatible restriction sites. If this cannot be done, it may be necessary to modify the termini of the DNA and/or vector by digesting back single-stranded DNA overhangs generated by restriction endonuclease cleavage to produce blunt ends, or to achieve the same result by filling in the single-stranded termini with an appropriate DNA polymerase.

Alternatively, desired sites may be produced, e.g., by ligating nucleotide sequences (linkers) onto the termini. Such linkers may comprise specific oligonucleotide sequences that define desired restriction sites. Restriction sites can also be generated by the use of the polymerase chain reaction (PCR). See, e.g., Saiki et al., Science 239:487 (1988). The cleaved vector and the DNA fragments may also be modified, if required, by homopolymeric tailing.

Recombinant expression vectors used in this invention are typically self-replicating DNA or RNA constructs comprising nucleic acids encoding MyD88 or fragments thereof, usually operably linked to suitable genetic control elements that are capable of regulating expression of the nucleic acids in compatible host cells. Genetic control elements may include a prokaryotic promoter system or a eukaryotic promoter expression control system, and typically include a transcriptional promoter, an optional operator to control the onset of transcription, transcription enhancers to elevate the level of mRNA expression, a sequence that encodes a suitable ribosome binding site, and sequences that terminate transcription and translation. Expression vectors also may contain an origin of replication that allows the vector to replicate independently of the host cell.

Vectors that could be used in this invention include microbial plasmids, viruses, bacteriophage, integratable DNA fragments, and other vehicles that may facilitate integration of the nucleic acids into the genome of the host. Plasmids are the most commonly used form of vector but all other forms of vectors which serve an equivalent function and which are, or become, known in the art are suitable for use herein. See, e.g., Pouwels et al., Cloning Vectors: A Laboratory Manual, 1985 and Supplements, Elsevier, N.Y., and Rodriguez et al. (eds.), Vectors: A Survey of Molecular Cloning Vectors and Their Uses, 1988, Buttersworth, Boston, Mass.

Expression of nucleic acids encoding MyD88 can be carried out by conventional methods in either prokaryotic or eukaryotic cells. Although strains of E. coli are employed most frequently in prokaryotic systems, many other bacteria, such as various strains of Pseudomonas and Bacillus, are known in the art and can be used as well.

Prokaryotic expression control sequences typically used include promoters, including those derived from the P-lactamase and lactose promoter systems [Chang et al., Nature, 198:1056 (1977)], the tryptophan (trp) promoter system [Goeddel et al., Nucleic Acids Res. 8:4057 (1980)], the lambda P_{L} promoter system [Shimatake et al., Nature, 292:128 (1981)] and the tac promoter [De Boer et al., Proc. Natl. Acad. Sci. USA 292:128 (1983)]. Numerous expression vectors containing such control sequences are known in the art and available commercially.

Suitable host cells for expressing nucleic acids encoding MyD88 or fragments thereof include prokaryotes and higher eukaryotes. Prokaryotes include both gram negative and positive organisms, e.g., E. coli and B. subtilis. Higher eukaryotes include established tissue culture cell lines from animal cells, both of non-mammalian origin, e.g., insect cells, and birds, and of mammalian origin, e.g., human, primates, and rodents.

Prokaryotic host-vector systems include a wide variety of vectors for many different species. As used herein, E. coli and its vectors will be used generically to include equivalent vectors used in other prokaryotes. A representative vector for amplifying DNA is pBR322 or many of its derivatives. Vectors that can be used to express MyD88 include, but are not limited to, those containing the lac promoter (pUC-series); trp promoter (pBR322-trp); Ipp promoter (the pIN-series); lambda-pP or pR promoters (pOTS); or hybrid promoters such as ptac (pDR540). See Brosius et al., “Expression Vectors Employing Lambda-, trp-, lac-, and Ipp-derived Promoters”, in Rodriguez and Denhardt (eds.) Vectors: A Survey of Molecular Cloning Vectors and Their Uses, 1988, Buttersworth, Boston, pp. 205-236.

Higher eukaryotic tissue culture cells are preferred hosts for the recombinant production of MyD88 or fragments thereof. Although any higher eukaryotic tissue culture cell line might be used, including insect baculovirus expression systems, mammalian cells are preferred. Transformation or transfection and propagation of such cells have become a routine procedure. Examples of useful cell lines include HeLa cells, Chinese hamster ovary (CHO) cell lines, baby rat kidney (BRK) cell lines, insect cell lines, bird cell lines, and monkey (COS) cell lines.

Expression vectors for such cell lines usually include an origin of replication, a promoter, a translation initiation site, RNA splice sites (if genomic DNA is used), a polyadenylation site, and a transcription termination site. These vectors also usually contain a selection gene or amplification gene. Suitable expression vectors may be plasmids, viruses, or retroviruses carrying promoters derived, e.g., from such sources as adenovirus, SV40, parvoviruses, vaccinia virus, or cytomegalovirus. Representative examples of suitable expression vectors include pCR®3.1, pCDNA1, pCD [Okayama et al., Mol. Cell Biol. 5:1136 (1985)], pMC1neo Poly-A [Thomas et al., Cell 51:503 (1987)], pUC19, pREP8, PSVSPORT and derivatives thereof, and baculovirus vectors such as pAC 373 or pAC 610.

A nucleic acid encoding MyD88 or a fragment thereof may be operably linked to a reporter gene. The term “operably linked” means that a nucleic acid encoding MyD88 or a fragment thereof and a reporter gene are so linked that expression of the nucleic acid encoding MyD88 or a fragment thereof also leads to expression of the reporter gene. Any reporter gene known in the art may be used in the method of the invention as long as it's expression is detectable. Examples of reporter genes include the luciferase gene, the chloramphenicol acetyl transferase (CAT) gene, the β-galactosidase gene, or a gene encoding a polyhistidine tag.

Vectors may be introduced into cells by any means known in the art. Examples of such means include transduction, transfection, electroporation and calcium phosphate precipitation.

Measuring the Expression Level of MvD88

Another step of the method of the invention involves measuring the expression level of MyD88. As is known in the art, there are many ways to measure the expression level of a protein. For example, an antibody or an antibody fragment may be used to bind to and measure the expression level of a protein. Also, cells may be lysed to determine the expression levels of a particular protein (by Western blot) or a particular RNA (by Northern blot). For example, a group of cells may be sacrificed to determine an average level of MyD88 expression, such as with a control cell in an experimental control. The level of MyD88 expression in cells that are contacted with a test sample can then be compared to the level of MyD88 expression in the experimental controls to identify an anti-cancer agent.

Antibody Production

Antigenic (i.e., immunogenic) fragments of MyD88, which may or may not have ligand binding activity, may be produced. Regardless of whether they have activity, such fragments, like the complete protein, are useful as antigens for preparing antibodies by standard methods. Shorter fragments can be concatenated or attached to a carrier. Because it is well known in the art that epitopes generally contain at least five, preferably at least 8, amino acid residues [Ohno et al., Proc. Nati. Acad. Sci. USA 82:2945 (1985)], fragments used for the production of antibodies will generally be at least that size. Preferably, they will contain even more residues. Whether a given fragment is immunogenic can readily be determined by routine experimentation.

Although it is generally not necessary when complete MyD88 protein is used as an antigen to elicit antibody production in an immunologically competent host, smaller antigenic fragments are preferably first rendered more immunogenic by cross-linking or concatenation, or by coupling to an immunogenic carrier molecule (i.e., a macromolecule having the property of independently eliciting an immunological response in a host animal). Cross-linking or conjugation to a carrier molecule may be required because small polypeptide fragments sometimes act as haptens (molecules which are capable of specifically binding to an antibody but incapable of eliciting antibody production, i.e., they are not immunogenic). Conjugation of such fragments to an immunogenic carrier molecule renders them more immunogenic through what is commonly known as the “carrier effect”.

Suitable carrier molecules include, e.g., proteins and natural or synthetic polymeric compounds, such as polypeptides, polysaccharides, lipopolysaccharides, etc. Protein carrier molecules are especially preferred, including, but not limited to, keyhole limpet hemocyanin and mammalian serum proteins, such as human or bovine gammaglobulin, human, bovine or rabbit serum albumin, or methylated or other derivatives of such proteins. Other protein-carriers will be apparent to those skilled in the art. Preferably, but not necessarily, the protein carrier will be foreign to the host animal in which antibodies against the fragments are to be elicited.

Covalent coupling to the carrier molecule can be achieved using methods well known in the art, the exact choice of which will be dictated by the nature of the carrier molecule used. When the immunogenic carrier molecule is a protein, the fragments of the invention can be coupled, e.g., using water-soluble carbodiimides, such as dicyclohexylcarbodiimide or glutaraldehyde.

Coupling agents such as these can also be used to cross-link the fragments to themselves without the use of a separate carrier molecule. Such cross-linking into aggregates can also increase immunogenicity. Immunogenicity can also be increased by the use of known adjuvants, alone or in combination with coupling or aggregation.

Suitable adjuvants for the vaccination of animals include, but are not limited to, Adjuvant 65 (containing peanut oil, mannide monooleate and aluminum monostearate); Freund's complete or incomplete adjuvant; mineral gels, such as aluminum hydroxide, aluminum phosphate and alum; surfactants, such as hexadecylamine, octadecylamine, lysolecithin, dimethyldioctadecylammonium bromide, N,N-dioctadecyl-N′,N′-bis(2-hydroxymethyl)propanediamine, methoxyhexadecylglycerol and pluronic polyols; polyanions, such as pyran, dextran sulfate, poly IC, polyacrylic acid and carbopol; peptides, such as muramyl dipeptide, dimethylglycine and tuftsin; and oil emulsions. The polypeptides could also be administered following incorporation into liposomes or other microcarriers.

Information concerning adjuvants and various aspects of immunoassays are disclosed, e.g., in the series by P. Tijssen, Practice and Theory of Enzyme Immunoassays, 3rd Edition, 1987, Elsevier, N.Y. Other useful references covering methods for preparing polyclonal antisera include Microbiology, 1969, Hoeber Medical Division, Harper and Row; Landsteiner, Specificity of Serological Reactions, 1962, Dover Publications, New York, and Williams, et al., Methods in Immunology and Immunochemistry, Vol. 1, 1967, Academic Press, N.Y.

Serum produced from animals immunized using standard methods can be used directly, or the IgG fraction can be separated from the serum using standard methods such as plasmaphoresis or adsorption chromatography with IgG-specific adsorbents such as immobilized Protein A. Alternatively, monoclonal antibodies can be prepared.

Hybridomas producing monoclonal antibodies against MyD88 or antigenic fragments thereof are produced by well-known techniques. Usually, the process involves the fusion of an immortalizing cell line with a B-lymphocyte that produces the desired antibody. Alternatively, non-fusion techniques for generating immortal antibody-producing cell lines can be used, e.g., virally-induced transformation [Casali et al., Science 234:476 (1986)]. Immortalizing cell lines are usually transformed mammalian cells, particularly myeloma cells of rodent, bovine, and human origin. Most frequently, rat or mouse myeloma cell lines are employed as a matter of convenience and availability.

Techniques for obtaining antibody-producing lymphocytes from mammals injected with antigens are well known. Generally, peripheral blood lymphocytes (PBLs) are used if cells of human origin are employed, or spleen or lymph node cells are used from non-human mammalian sources. A host animal is injected with repeated dosages of the purified antigen (human cells are sensitized in vitro), and the animal is permitted to generate the desired antibody-producing cells before they are harvested for fusion with the immortalizing cell line. Techniques for fusion are also well known in the art, and in general involve mixing the cells with a fusing agent, such as polyethylene glycol.

Hybridomas are selected by standard procedures, such as HAT (hypoxanthine-aminopterin-thymidine) selection. Those secreting the desired antibody are selected using standard immunoassays, such as Western blotting, ELISA (enzyme-linked immunosorbent assay), RIA (radioimmunoassay), or the like. Antibodies are recovered from the medium using standard protein purification techniques [Tijssen, Practice and Theory of Enzyme Immunoassays (Elsevier, Amsterdam, 1985)].

Many references are available to provide guidance in applying the above techniques [Kohler et al., Hybridoma Techniques (Cold Spring Harbor Laboratory, N.Y., 1980); Tijssen, Practice and Theory of Enzyme Immunoassays (Elsevier, Amsterdam, 1985); Campbell, Monoclonal Antibody Technology (Elsevier, Amsterdam, 1984); Hurrell, Monoclonal Hybridoma Antibodies: Techniques and Applications (CRC Press, Boca Raton, Fla. 1982)]. Monoclonal antibodies can also be produced using well-known phage library systems. See, e.g., Huse, et al., Science 246:1275 (1989); Ward, et al., Nature, 341:544 (1989).

Antibodies thus produced, whether polyclonal or monoclonal, can be used, e.g., in an immobilized form bound to a solid support by well known methods, to purify the protein by immunoaffinity chromatography.

Antibodies against the antigenic fragments can also be used, unlabeled or labeled by standard methods, as the basis for immunoassays of MyD88. The particular label used will depend upon the type of immunoassay. Examples of labels that can be used include, but are not limited to, radiolabels, such as ³²P, ¹²⁵I, ³H and ¹⁴C; fluorescent labels, such as fluorescein and its derivatives, rhodamine and its derivatives, dansyl and umbelliferone; chemiluminescers, such as luciferia and 2,3-dihydrophthalazinediones; and enzymes, such as horseradish peroxidase, alkaline phosphatase, lysozyme and glucose-6-phosphate dehydrogenase.

The antibodies can be tagged with such labels by known methods. For example, coupling agents, such as aldehydes, carbodiimides, dimaleimide, imidates, succinimides, bisdiazotized benzadine and the like may be used to tag the antibodies with fluorescent, chemiluminescent or enzyme labels. The general methods involved are well known in the art and are described, e.g., in Immunoassay: A Practical Guide, 1987, Chan (Ed.), Academic Press, Inc., Orlando, Fla. Such immunoassays could be carried out, for example, on fractions obtained during purification of the proteins.

The antibodies of the present invention can also be used to identify particular cDNA clones expressing MyD88 in expression cloning systems.

Neutralizing antibodies specific for MyD88 can also be used as antagonists (inhibitors) to block the protein. Such neutralizing antibodies can readily be identified through routine experimentation, e.g., by using the radioligand binding assay described infra. Binding to MyD88 can be accomplished using complete antibody molecules, or well-known antigen binding fragments such as Fab, Fc, F(ab)₂, and Fv fragments.

Definitions of such fragments can be found, e.g., in Klein, Immunology (John Wiley, New York, 1982); Parham, Chapter 14, in Weir, ed. Immunochemistry, 4th Ed. (Blackwell Scientific Publishers, Oxford, 1986). The use and generation of antibody fragments has also been described, e.g.: Fab fragments [Tijssen, Practice and Theory of Enzyme Immunoassays (Elsevier, Amsterdam, 1985)], Fv fragments [Hochman et al., Biochemistry 12:1130 (1973); Sharon et al., Biochemistry 15:1591 (1976); Ehrlich et al., U.S. Pat. No. 4,355,023] and antibody half molecules (Auditore-Hargreaves, U.S. Pat. No. 4,470,925). Methods for making recombinant Fv fragments based on known antibody heavy and light chain variable region sequences have further been described, e.g., by Moore et al. (U.S. Pat. No. 4,642,334) and by Plückthun [Bio/Technology 9:545 (1991)]. Alternatively, they can be chemically synthesized by standard methods.

Anti-idiotypic antibodies, both polyclonal and monoclonal, can also be produced using the antibodies elicited against the protein as antigens. Such antibodies can be useful as they may mimic the protein.

Future Developments

The invention relates to methods for identifying anti-cancer agents using MyD88 as a therapeutic target. Based upon the below examples, we propose the use of MyD88 expression inhibitors, such as siRNA, ribozymes or antisense nucleic acids, or function inhibitors, such as small molecules or organic compounds, as therapeutic agents for cancer therapy. Once a suitable agent is identified, it may be formulated into a pharmaceutical composition for administration to a patient. In addition, the effectiveness of an agent in preventing or treating cancer may be improved by administering the agent in combination with a chemotherapeutic compound or treatment that is effective for the same purpose. A treatment for cancer includes surgery, to remove a cancer, and radiation treatment, to reduce or kill a cancer or tumor. Alternatively, the effectiveness of an agent in preventing or treating cancer may be improved by administering the agent in combination with other modalities, such as, for example, a farnesyl transferase inhibitor.

### EXAMPLES

The present invention may be better understood by reference to the following non-limiting examples, which are provided as exemplary of the invention. The following examples are presented in order to more fully illustrate the invention, and should in no way be construed as limiting the broad scope of the invention. Unless otherwise indicated, percentages given below for solids in solid mixtures, liquids in liquids, and solids in liquids are on a wt/wt, vol/vol and wt/vol basis, respectively. Sterile conditions were generally maintained during cell culture.

### Materials and General Methods

Standard methods were used, as described, e.g., in Maniatis et al., Molecular Cloning: A Laboratory Manual, 1982, Cold Spring Harbor Laboratory, Cold Spring Harbor Press; Sambrook et al., Molecular Cloning: A Laboratory Manual, (2d ed.), Vols 1-3, 1989, Cold Spring Harbor Press, NY; Ausubel et al., Biology, Greene Publishing Associates, Brooklyn, N.Y.; or Ausubel, et al. (1987 and Supplements), Current Protocols in Molecular Biology, Greene/Wiley, New York; Innis et al. (eds.) PCR Protocols: A Guide to Methods and Applications, 1990, Academic Press, N.Y.

### Example 1

In these sets of experiments, we subjected wild-type (wt), MyD88−/− and IL-1R−/− mice to a DMBA/TPA two-stage, ras-dependent skin carcinogenesis protocol.

Wild type C56BL/6 mice were obtained from Charles River Laboratories (L'arbresle, France). MyD88−/− mice were obtained from Dr. Shizuo Akira at Osaka University in Japan. IL-1R−/− mice were purchased from the Jackson Laboratory (Bar Harbor, Me.).

For two-stage chemical carcinogenesis, the backs of 8-week-old mice were shaved and treated with a single application of 7,12-dimethylbenz[a]anthracene (DMBA, 25 μG in 200 μl acetone; Sigma, Lyon, France), followed by biweekly applications of 12-O-tetradecanoylphorbol-13-acetate (TPA, 200 μl of 10⁻⁴ M solution in acetone; Sigma, Lyon, France) for 20 weeks. Mice were visually examined at least twice per week and tumors per mouse were counted. Mice were sacrificed if moribund, if any individual tumor reached a diameter of 1 cm, or at the termination of the experiment. These experiments addressed ras-mediated cancers because all of the protocols that were used were ras-dependent.

The results of these experiments show that, after 20 weeks, almost no MyD88−/− mice developed tumors, whereas about 85% of the wt mice and about 40% of the IL-1R−/− mice developed tumors. Specifically, the percentage of wt mice that developed tumors over time was as follows: 0% for 0-8 weeks, 15% at 9 weeks, 35% at 10-11 weeks, 40% at 12 weeks, 50% at 13 weeks, 70% at 14-15 weeks, 80% at 16 weeks, and 85% at 17-20 weeks. The percentage of IL-1R−/− mice that developed tumors over time was as follows: 0% for 0-10 weeks, 10% at 11 weeks, 20% at 12-13 weeks, 30% at 14-17 weeks and 40% at 18 weeks. The percentage of MyD88−/− mice that developed tumors over time was as follows: 0% at 0-14 weeks and 5% at 15-20 weeks.

It is apparent that almost no MyD88−/− mice developed tumors. Furthermore, the resistance of the MyD88−/− mice was not due to an absence of IL-1 signaling because the IL-1R−/− mice, while less susceptible than wt mice to tumor development, did develop skin tumors in response to DMBA/TPA.

### Example 2

The data in the above example suggested another, non-inflammatory function for MyD88 in ras-mediated tumor development. To address whether MyD88 acts in a cell-autonomous fashion, we generated mouse embryo fibroblasts (MEFs) from both wt and MyD88−/− mice.

For isolation of MEFs, day E14 embryos were harvested from pregnant wild-type or MyD88−/− mice and placed in DMEM (Gibco). Ten embryos were used to allow for collection of a sufficient quantity of cells for experimentation. The head and liver were removed, and the carcasses were washed three-times in DMEM. The embryos were then minced with a sterile razor blade, placed in 5 ml trypsin:versene (1:1), and incubated for 20 min at 37° C. The cells were collected in media with 10% serum, then centrifuged and resuspended in 50 ml DMEM plus 10% FCS, and seeded onto five 10-cm tissue culture dishes containing DMEM plus 10% FCS. The medium was changed the following day, and the cells were grown until confluent, then split 1:10 and grown again to confluency. The MEFs were grown in culture media for 3 days and then pulsed for 60 minutes with BrdU. Following fixation, permeabilization, and staining with FITC-labeled anti-BrdU mAb, the cells were analyzed by FACS. Exponentially growing cells were incubated in culture medium containing 1 μg/ml Bromodeoxyuridine (BrdU) for 1 h. The labeled cells were washed twice with 1× PBS and then fixed 30 min with 70% ethanol. The fixed cells were treated with 1 M HCl for 20 min; neutralized by 1 M borax for 3 min and washed twice with PBS/2.5% FCS. Cells were stained with anti BrdU-FITC antibody (BD Biosciences) and propidium iodide (PI) for cell cycle analysis.

The data show that the percentage of wt MEFs that incorporated BrdU was 40% while only 26% of the MyD88−/− MEFs incorporated BrdU.

These data indicate that MyD88−/− MEFs proliferated somewhat more slowly than wt MEFs. Therefore, the absence of MyD88 decreases the proliferation rate of MEFs.

### Example 3

We generated mouse embryo fibroblasts (MEFs) from both wt and MyD88−/− mice, as described above. Both wt and MyD88−/− MEFs were treated once with DMBA and then twice a week with TPA for three weeks. Control MEFs (wt and MyD88−/−) were untreated. The plates were then fixed with 4% paraformaldehyde, colored with 0.1% crystal violet, and foci were observed.

The results show that a moderate number of foci developed on the untreated wt MEF plates whereas a large number of foci developed on the treated wt MEF plates. In contrast, both the untreated and treated MyD88−/− MEFs showed that no foci developed.

These data indicate that MyD88−/− MEFs were completely resistant to cell transformation in vitro by DMBA/TPA. Therefore, the absence of MyD88 increases the resistance of MEFs to cell transformation in vitro by DMBA/TPA.

### Example 4

We generated mouse embryo fibroblasts (MEFS) from both wt and MyD88−/− mice, as described above. Both wt and MyD88−/− MEFs were transfected with ras and myc and then plated at 3×10⁵ cells/10 cm on culture dishes. After 14 days, the dishes were fixed with 4% paraformaldehyde, colored with 0/1% crystal violet, and foci were counted.

The results show that the wt MEFs developed 14 foci/dish whereas the MyD88−/− MEFs developed 0.25 foci/dish.

These data indicate that MyD88−/− MEFs were completely resistant to transfection with ras and myc. Therefore, the absence of MyD88 increases the resistance of MEFs to transfection with ras and myc.

We claim:

1) A method for identifying an anti-cancer agent comprising:
a) contacting a test sample with a cell that expresses MyD88 or a biologically active fragment thereof; and
b) measuring MyD88 expression level in said cell,
whereby the test sample is identified to contain an anti-cancer agent if the test sample causes decreased MyD88 expression when compared to a control cell.

2) The method of claim 1 wherein the contacting step comprises introducing the sample into the cell.

3) A method for identifying an anti-cancer agent comprising:
a) contacting a test sample with a cell having a vector comprising a nucleic acid encoding MyD88 or a biologically active fragment thereof; and
b) measuring MyD88 expression level in said cell,
whereby the test sample is identified to contain an anti-cancer agent if the test sample causes decreased MyD88 expression when compared to a control cell.

4) The method of claim 3 wherein the contacting step comprises introducing the sample into the cell.

5) A method for identifying an anti-cancer agent comprising:
a) contacting a test sample with a cell having a vector comprising a nucleic acid encoding MyD88 or a biologically active fragment thereof that is operably linked to a reporter gene; and
b) measuring MyD88 expression level in said cell,
whereby the test sample is identified to contain an anti-cancer agent if the test sample causes decreased MyD88 expression when compared to a control cell.

6) The method of claim 5 wherein the contacting step comprises introducing the sample into the cell.
